(19) United States
(12) Patent Application Publication
(10) Pub. No.: US 2022/0160319 A1
(43) Pub. Date: May 26, 2022

(54) APPARATUS, SYSTEM, METHOD, AND STORAGE MEDIUM

(71) Applicant: CANON KABUSHIKI KAISHA, Tokyo (JP)

(72) Inventors: Daisuke Yamada, Kanagawa (JP); Kazuma Obara, Tokyo (JP)

(21) Appl. No.: 17/521,733

(22) Filed: Nov. 8, 2021

(30) Foreign Application Priority Data
Nov. 20, 2020 (JP) 2020-192933

(51) Int. Cl. A61B 6/00; G06T 7/00

(52) U.S. Cl. CPC A61B 6/5247; G06T 7/0012; G06T 2207/30008; A61B 6/505; G06T 2207/20224; A61B 6/482

(57) ABSTRACT

An apparatus includes an acquisition unit configured to acquire a first image and a second image obtained by detecting radiation of different energies, and a subtraction image generated using the first image and the second image, and an output control unit configured to control output of the first image, the second image, and the subtraction image based on examination information.

### BACKGROUND

### Field of the Disclosure

The aspect of the embodiments relates to an image processing apparatus, an image processing system, an image processing method, and a storage medium.

### Description of the Related Art

A radiographing apparatus using a flat panel detector (FPD) made of a semiconductor material is widely used in medical image diagnosis and non-destructive examination. As one of image capturing methods using the FPD, a method of acquiring a subtraction image using radiation of different energies is known.

The subtraction image is generated by performing energy subtraction processing of performing subtraction while applying weights on a plurality of radiographic images captured with radiation of different energies. As a method for capturing a plurality of radiographic images, in addition to a method of acquiring one radiographic image by one time of image capturing, according to a configuration of the FPD of Japanese Patent Application Laid-Open No. 2001-249182, it is possible to simultaneously capture two radiographic images by one time of irradiation.

Japanese Patent Application Laid-Open No. 2002-216107 describes that capacity of a medical image management system does not become tight by compressing each of captured radiographic images and subtraction images, and outputting the compressed images to an outside of a radiographing apparatus, for example, to a medical image management system such as a picture archiving and communication system (PACS).

In general, a radiographic image captured by a radiographing apparatus is output to a medical image management system such as the PACS unless image capturing is handled as a failure. In a similar manner, in the image capturing for acquiring a subtraction image, a plurality of radiographic images (an image captured by radiation of a relatively high energy will be referred to as a high energy image, and an image captured by radiation of a relatively low energy will be referred to as a low energy image) captured by radiation of different energies and the subtraction image are output to the medical image management system such as the PACS.

However, in the image capturing for acquiring the subtraction image, there may be an image unnecessary for diagnosis out of the high energy image and the low energy image depending on examination information. For example, in a case where a soft tissue image from which bone has been removed is generated as the subtraction image, which is to be used in diagnosis at a respiratory department, a low-energy image may be unnecessary. In a case where a bone image emphasizing bone for orthopedic use is generated as the subtraction image, a high energy image may be unnecessary.

### SUMMARY

According to an aspect of the embodiments, an apparatus includes an acquisition unit configured to acquire a first image and a second image obtained by detecting radiation of different energies, and a subtraction image generated using the first image and the second image, and a control unit configured to control output of the first image, the second image, and the subtraction image based on examination information.

Further features of the disclosure will become apparent from the following description of exemplary embodiments with reference to the attached drawings.

### BRIEF DESCRIPTION OF THE DRAWINGS

FIG. 1 is a diagram illustrating a configuration example of a radiographing system according to a first exemplary embodiment.

FIG. 2 is a flowchart illustrating an example of image capturing processing according to an exemplary embodiment of the disclosure.

FIG. 3 is a diagram illustrating an example of examination information according to an exemplary embodiment of the disclosure.

FIG. 4 is a flowchart illustrating an example of processing of an output control unit according to the first exemplary embodiment.

FIG. 5 is a diagram illustrating an example of an output type table to be referred to by the output control unit according to the first exemplary embodiment.

FIG. 6 is a diagram illustrating a configuration example of a radiographing system according to a second exemplary embodiment.

FIG. 7 is a flowchart illustrating an example of processing of an output control unit and an information providing unit according to the second exemplary embodiment.

FIG. 8 is a diagram illustrating an example of an output type table to be referred to by the output control unit and the information providing unit according to the second exemplary embodiment.

FIG. 9 is a diagram illustrating an example of an output type table to be referred to by the output control unit and the information providing unit according to the second exemplary embodiment.

### DESCRIPTION OF THE EMBODIMENTS

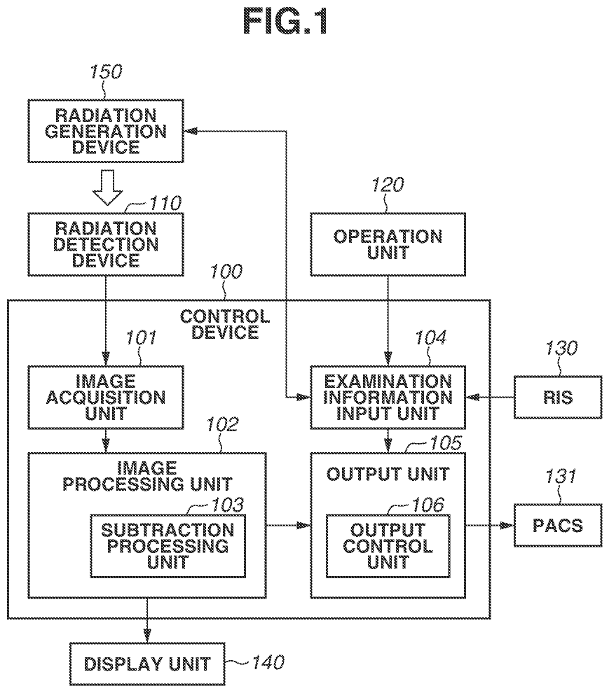

FIG. 1 is a diagram illustrating a configuration example of a radiographing system according to a first exemplary embodiment. The radiographing system, which is a system for obtaining a radiographic image using an energy subtraction method, includes a control device 100, a radiation detection device 110, an operation unit 120, a radiology information system (RIS) 130, a medical image management system (a picture archiving and communication system (PACS)) 131, a display unit 140, and a radiation generation device 150. The control device 100, to which the RIS 130 and the PACS 131 are connected, controls radiography using the radiation detection device 110 and the radiation generation device 150.

The radiation detection device 110 detects radiation, which is emitted from the radiation generation device 150 and passes through a subject (not illustrated), and outputs image data according to the radiation. The energy subtraction method is a method of obtaining a new radiographic image (for example, bone and soft tissue images) from a plurality of radiographic images obtained by a plurality of times of image capturing in which radiation having different energies is emitted from the radiation generation device 150. In addition, there is also a radiation detector capable of detecting radiation of a plurality of different energies by one time of irradiation and obtaining a radiographic image. The image data can also be referred to as, for example, an image, a medical image, or a radiographic image. Specifically, the radiation detection device 110 detects the radiation, which has transmitted the subject, as an electric charge corresponding to the amount of transmitted radiation. For example, as the radiation detection device 110, a direct conversion sensor that directly converts radiation into charges, such as amorphous selenium (a-Se) that converts radiation into charges, or an indirect sensor using a scintillator such as cesium iodide (CsI) that converts radiation into visible light and a photoelectric conversion element such as amorphous silicon (a-Si), is used.

Further, the radiation detection device 110 generates image data by performing A/D conversion on the detected charges, and outputs the image data to the control device 100.

The control device 100 is connected to the radiation detection device 110 via, for example, a wired or wireless network or a leased line. The radiation detection device 110 captures an image of the radiation generated by the radiation generation device 150 and outputs image data to the control device 100. The control device 100 has an application function that operates on a computer. In other words, the control device 100 includes one or more processors and a memory, and implements each functional unit described below by the processor executing a program stored in the memory. However, a part or all of the functional units can be implemented by dedicated hardware. The control device 100 performs image processing on the image data output from the radiation detection device 110, generates an image, and displays the image on the display unit 140. The operation unit 120 receives an instruction from an operator. In addition, the control device 100 has a function of controlling each component. The control device 100 outputs an image to the display unit 140 and provides a graphical user interface using the display unit 140 while controlling an operation of the radiation detection device 110.

Further, the control device 100 controls timings of generating radiation at a radiation generation unit and image capturing conditions of radiation. Furthermore, the control device 100 includes an image acquisition unit 101 and controls a timing at which image data of the radiation detection device 110 is captured and a timing at which the image data is output.

The control device 100 includes an image processing unit 102 that performs image processing such as noise reduction and gradation processing on the image data output from the radiation detection device 110. The image processing unit 102 performs image processing such as trimming or rotation on the image output from the radiation detection device 110.

The control device 100 includes a subtraction processing unit 103. The subtraction processing unit 103 performs subtraction processing of performing subtracting while applying weights on a plurality of radiographic images captured with different energies.

The control device 100 includes an examination information input unit 104 for allowing the operator to manually input examination information through the operation unit 120 or for allowing the operator to select the examination information acquired from the RIS 130 through the operation unit 120. The examination information input to the examination information input unit 104 is managed in association with the image data captured by the radiation detection device 110.

The control device 100 includes an output unit 105 that converts the image output from the image processing unit 102 into a Digital Imaging and Communications in Medicine (DICOM) format and outputs the image to the PACS 131. As a feature of the present exemplary embodiment, the output unit 105 includes an output control unit 106. The output control unit 106 controls output of a high energy image, a low energy image, and a subtraction image generated from the high energy image and the low energy image.

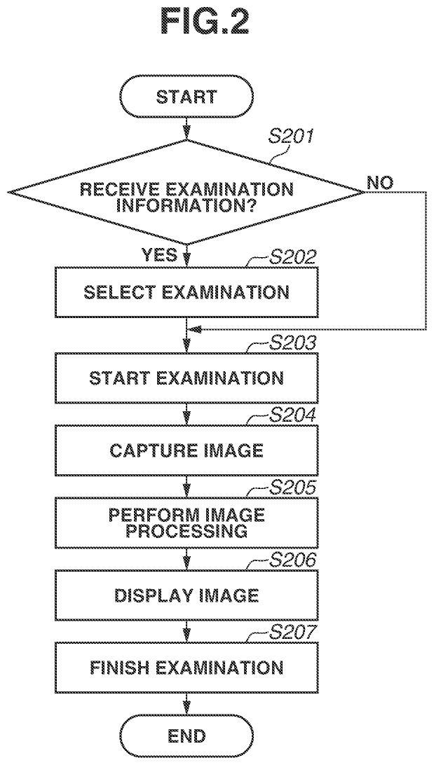

Subtraction image capturing processing according to the exemplary embodiment will be described with reference to a flowchart of FIG. 2.

### <Step S201: Input Examination Information>

In step S201, the examination information input unit 104 receives an instruction from the operator and selects, as a method of inputting the examination information, whether to use the examination information received from the RIS 130 or to allow the operator to manually input the examination information. In a case where the former method is selected (YES in step S201), the processing proceeds to step S202. On the other hand, in a case where the latter method is selected (NO in step S201), the manual input of the examination information from the operator is received, and then the processing proceeds to step S203.

### <Step S202: Select Examination>

In step S202, the examination information input unit 104 sets one of the examination information received from the RIS 130 as an examination target. In such processing, for example, the examination information is set as an image capturing target according to an operation input in which the operator selects one of the plurality of pieces of examination information displayed in a list form.

### <Step S203: Start Examination>

In step S203, the examination information is determined by the operation input from the operator, and the control device 100 transmits a signal for causing a state to transition to a preparation state to the radiation detection device 110 related to image capturing. In response to this, in a case where a bias voltage is not applied to a two-dimensional image pickup element, the radiation detection device 110 controls a bias power supply by a main control circuit and applies the bias voltage to the two-dimensional image pickup element. Thereafter, in order to read out a dark current signal accumulated in a pixel, a drive circuit performs initialization to read out an image signal from a pixel array. After the initialization completes, the radiation detection device 110 transmits state information indicating that the radiation detection device 110 is ready for obtaining a radiographic image to the control device 100.

### <Step S204: Capture Image>

In step S204, the image acquisition unit 101 acquires a radiographic image generated by the drive circuit of the radiation detection device 110 detecting the emitted radiation and a readout circuit reading out an image signal obtained by the detection. Thereafter, the radiation detection device 110 transmits the radiographic image to the control device 100. Accordingly, the image acquisition unit 101 of the control device 100 acquires the radiographic image.

### <Step S205: Image Processing>

In step S205, the image processing unit 102 performs image processing such as noise reduction and gradation processing in the image processing unit 102 on the radiographic image acquired by the image acquisition unit 101.

After both the high energy image and the low energy image are acquired, in step S205, the subtraction processing unit 103 in the image processing unit 102 performs subtraction while applying weights on the respective radiographic images and outputs the subtraction image.

### <Step S206: Display Image>

In step S206, the image processing unit 102 displays the radiographic image subjected to the image processing in step S205 on the display unit 140. In a case where the subtraction image is output in step S205, the subtraction image is further displayed on the display unit 140.

<Step S207: Finish examination>

In step S207, the control device 100 finishes the examination by an input operation by the operator. Processing of outputting the image to the PACS 131, which is an external device, is performed at a timing of the end of the examination. At this time, the output control unit 106 in the output unit 105 performs an operation of selectively outputting the output image based on the examination information, which will be described in detail below. The timing at which the output unit 105 performs the operation may not be the timing of the end of the examination and can be, for example, a case where the image is re-output anew after the examination ends.

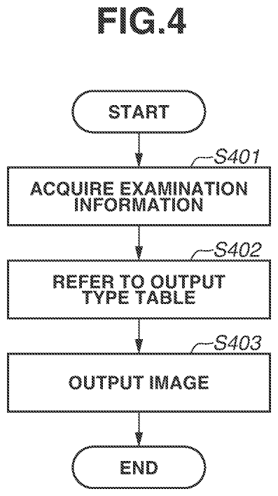

Processing of the output control unit 106 in the output unit 105 according to the exemplary embodiment will be described with reference to the flowchart of FIG. 4.

### <Step S401: Acquire Examination Information>

In step S401, the output control unit 106 acquires the examination information input at the start of image capturing. The examination information can be loaded on a memory or can be held in a storage device such as a database or a file (not illustrated) and read from the storage device in step S401.

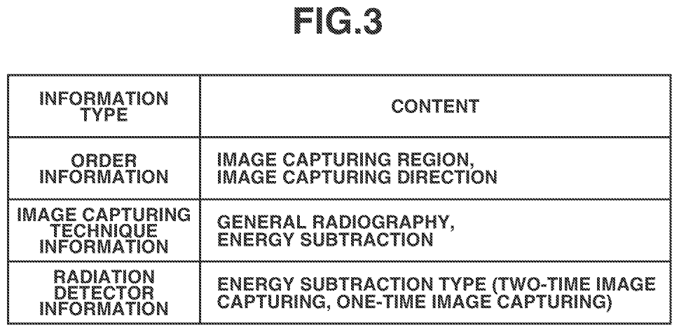

The examination information includes, for example, at least one of order information, image capturing technique information, and radiation detector information illustrated in FIG. 3. For example, the order information includes information on at least one of an image capturing region and an image capturing direction of radiography. The image capturing technique information includes attributes (for example, general radiography, energy subtraction) of the technique. Further, the radiation detector information includes whether the radiation detector is a model capable of acquiring radiographic images of different energies by two times of image capturing or a model capable of acquiring radiographic images of different energies by one time of image capturing. In other words, the radiation detector information includes information as to a method for acquiring a first radiographic image and a second radiographic image. In addition, although not illustrated, information indicating use of the subtraction image can be included. The information indicating use of the subtraction image includes, for example, information indicating whether the subtraction image is used for observing soft tissue or bone. For example, in a case where the use of the subtraction image can be specified from information such as the order information, the image capturing technique information, and the radiation detector information, these pieces of information can be used as the information indicating the use of the subtraction image. More specifically, for example, in a case where use of diagnosis of the subtraction image is specified from information regarding at least one of the image capturing region and the image capturing direction of radiography included in the order information, the information can be used as the information indicating the use of the subtraction image.

### <Step S402: Refer to Output Type Table>

In step S402, the output unit 105 acquires an output type table in which the examination information and whether to output each of the high energy image, the low energy image, and the subtraction image to the medical image management system are defined in association with each other. In other words, the output control unit 106 corresponds to an example of an output control unit that acquires output information in which the examination information is associated with information indicating which image of the first radiographic image, the second radiographic image, and the subtraction image is to be output.

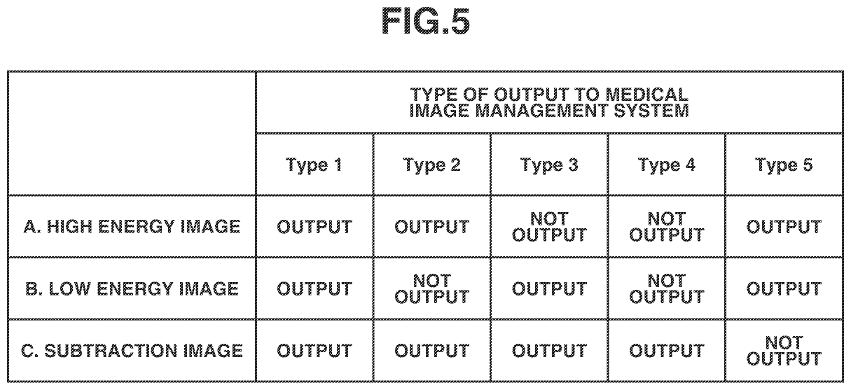

An example of the output type table acquired by the output control unit 106 is illustrated in FIG. 5. In this table, for example, the examination information includes information regarding the image capturing region, information regarding the image capturing direction, and the image capturing technique information, and association is defined to be Type 2 in advance in a case where the image capturing region is a chest part, the image capturing direction is a front side, and the image capturing technique information is energy subtraction. As another example, association is defined to be Type 3 in advance in a case where the image capturing region is four limbs, the image capturing direction is a front side, and the image capturing technique information is energy subtraction.

In another example, in a case where the examination information includes information indicating that the subtraction image is used for observing soft tissue, the low-energy image is not required for diagnosis, and thus, association with Type 2 is defined. Further, in another example, in a case where the examination information includes information indicating that the subtraction image is used for observing the bone portion, the high energy image is not required for diagnosis, and thus, association with Type 3 is defined.

Association between the above-described examination information and whether to output each of the high energy image, the low energy image, and the subtraction image to the medical image management system is an example, and the association is not limited thereto.

### <Step S403: Output Image>

In step S403, the output control unit 106 controls output of the high energy image, the low energy image, and the subtraction image to the medical image management system according to the output type table acquired in step S402.

As described above, a series of processing of the radiographing system according to the present exemplary embodiment is performed.

According to the present exemplary embodiment, it is possible to appropriately control output of an image in image capturing for acquiring a subtraction image and prevent storage capacity of a medical image management system such as a PACS from becoming tight.

In particular, by selectively outputting the high energy image, the low energy image, and the subtraction image to the medical image management system based on the examination information, it is possible to improve efficiency of radiographic interpretation and prevent storage capacity of the medical image management system such as the PACS from becoming tight.

### MODIFIED EXAMPLES

In the first exemplary embodiment, an example of selectively outputting the high energy image, the low energy image, and the subtraction image to the medical image management system based on the examination information has been described. However, output of the image can be controlled not based on the examination information. For example, an image to be output is selected depending on whether the subtraction image is an image in which soft tissue is emphasized or an image in which a bone portion is emphasized. More specifically, for example, in a case where the subtraction image is an image in which the soft tissue is emphasized, at least the high energy image and the subtraction image are output. In a case where the subtraction image is an image in which the bone portion is emphasized, at least the low energy image and the subtraction image are output.

In this case, for example, a determination unit (not illustrated) included in the control device 100 determines which part is emphasized in the subtraction image. Then, as a result of the determination, in a case where the subtraction image is an image in which the soft tissue is emphasized, at least the high energy image and the subtraction image are output. In a case where the subtraction image is an image in which the bone portion is emphasized, at least the low energy image and the subtraction image are output. The determination can be performed using information attached to the subtraction image, or can be performed through image processing, or the like.

As a result, it is possible to selectively output the high energy image, the low energy image, and the subtraction image to the medical image management system based on the information obtained from the subtraction image without using the examination information.

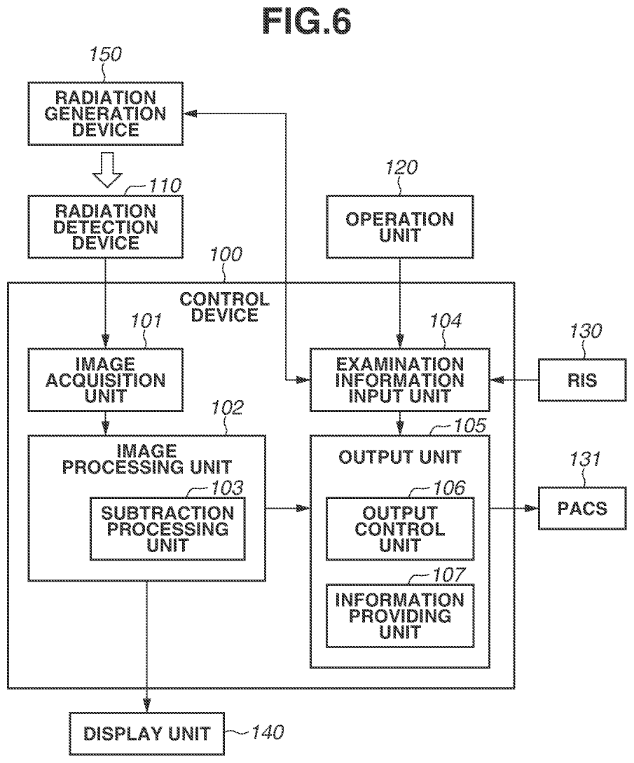

FIG. 6 illustrates a second exemplary embodiment in which the output unit 105 having the configuration described in the first exemplary embodiment includes an information providing unit 107. In the first exemplary embodiment, an example of selectively outputting the high energy image, the low energy image, and the subtraction image to the medical image management system based on the examination information has been described. However, for example, in a case where the high energy image or the low energy image unnecessary for diagnosis is not output to the medical image management system, there is an issue that dosage information attached to a captured image is not output to the medical image management system, so that dosage cannot be appropriately managed for each captured image.

Thus, the present exemplary embodiment describes a mode in which supplementary information of an image that is not output to the medical image management system, out of the high energy image and the low energy image, is attached to the subtraction image.

This exemplary embodiment is an example of a radiation detector that obtains radiographic images of different energies by two times of irradiation. Processing other than that of the output unit 105 is the same as that in the first exemplary embodiment, and thus the description thereof will be omitted.

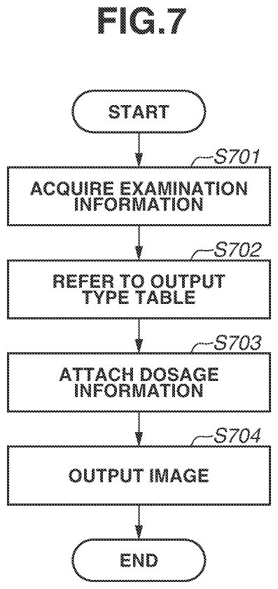

Processing of the information providing unit 107 included in the output unit 105 according to the exemplary embodiment will be described with reference to the flowchart of FIG. 7.

### <Step S701: Acquire Examination Information>

In step S701, the information providing unit 107 acquires the examination information input at the start of image capturing. The examination information can be loaded on a memory or can be held in a storage device such as a database or a file (not illustrated) and read from the storage device in step S701.

### <Step S702: Refer to Output Type Table>

In step S702, the output unit 105 acquires an output type table in which it is determined whether to output each of the high energy image, the low energy image, and the subtraction image to the medical image management system according to the information included in the examination information. The output type table acquired in step S702 includes information on dosage information to be attached to the subtraction image in addition to the output type table illustrated in FIG. 5 described in the first exemplary embodiment.

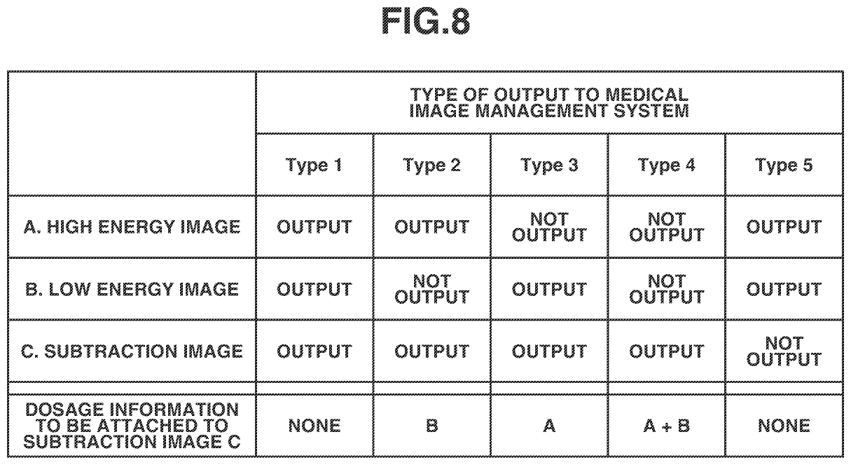

An example of the output type table acquired by the output unit 105 is illustrated in FIG. 8. In this table, for example, the examination information includes information regarding the image capturing region, information regarding the image capturing direction, and the image capturing technique information, and association is defined to be Type 2 in advance in a case where the image capturing region is a chest part, the image capturing direction is a front side, and the image capturing technique information is energy subtraction. As another example, association is defined to be Type 3 in advance in a case where the image capturing region is four limbs, the image capturing direction is a front side, and the image capturing technique information is energy subtraction.

In another example, in a case where the examination information includes information indicating that the subtraction image is used for observing soft tissue, the low-energy image is not required for diagnosis, and thus, association with Type 2 is defined. Further, in another example, in a case where the examination information includes information indicating that the subtraction image is used for observing the bone portion, the high energy image is not required for diagnosis, and thus, association with Type 3 is defined.

Association between the above-described examination information and whether to output each of the high energy image, the low energy image, and the subtraction image to the medical image management system is an example, and the association is not limited thereto.

### <Step S703: Attach Dosage Information>

In step S703, the information providing unit 107 attaches the dosage information to the subtraction image according to the output type table acquired in step S702. For example, in a case of Type 2 in which the low energy image is not output, the information providing unit 107 attaches the dosage information of the low energy image (B in FIG. 8) to the subtraction image. In another example, in a case of Type 3 in which the high energy image is not output, the information providing unit 107 attaches the dosage information of the high energy image (A in FIG. 8) to the subtraction image. In addition, in another example, in a case of Type 4 in which neither the high energy image nor the low energy image is output, the information providing unit 107 attaches the dosage information of the high energy image and the low energy image (A+B in FIG. 8).

The method for attaching the dosage information is not limited, but in a case where an image is output in a DICOM format, the information is stored in a DICOM tag.

### <Step S704: Output Image>

In step S704, the output control unit 106 controls output of the high energy image, the low energy image, and the subtraction image to the medical image management system according to the output type table acquired in step S702.

As described above, a series of processing of the radiographing system according to the present exemplary embodiment is performed.

According to the present exemplary embodiment, it is possible to attach, to the subtraction image, supplementary information of an image that is not output to the medical image management system, out of the high energy image and the low energy image. As a result, the supplementary information of the image that is not output to the medical image management system such as the PACS is attached to the subtraction image, so that the dosage can be managed for each captured image on the medical image management system such as the PACS.

A third exemplary embodiment is an example of using a radiation detector that obtains radiographic images of different energies by one time of irradiation in the configuration described in the second exemplary embodiment. An output type table acquired in step S702 will be described as a difference from the second exemplary embodiment. A series of processing is similar to that of the second exemplary embodiment, and thus description thereof will be omitted.

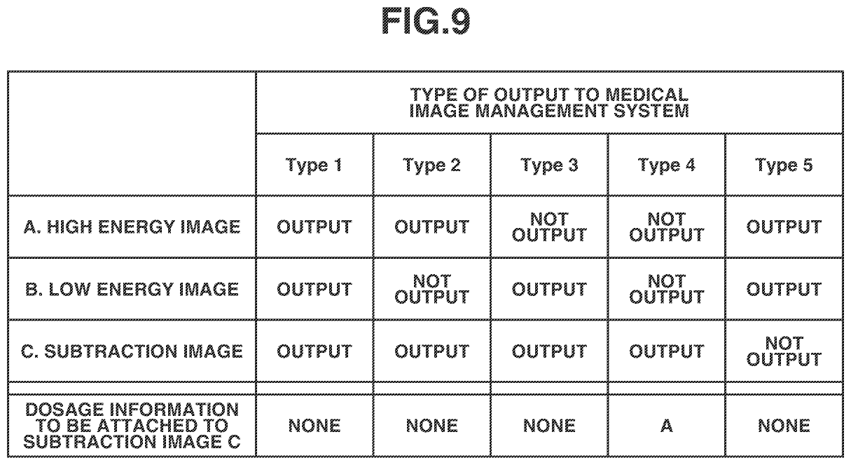

An example of the output type table in the present exemplary embodiment is illustrated in FIG. 9. In a case where radiographic images of different energies can be obtained by one time of irradiation, there is dosage information corresponding to one time. Thus, in a case where either the high energy image or the low energy image is output to the medical image management system as in Types 1, 2, 3, and 5, the information providing unit 107 does not provide the dosage information to the subtraction image.

On the other hand, in a case of Type 4 in which neither the high energy image nor the low energy image is output to the medical image management system, the information providing unit 107 attaches the dosage information (A in FIG. 9).

According to the present exemplary embodiment, even in a case where the high energy image and the low energy image are not output to the medical image management system, it is possible to manage the dosage for each captured image on the medical image management system such as the PACS by attaching the supplementary information of the image that is not output, to the subtraction image.

### OTHER EMBODIMENTS

Embodiment(s) of the disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2020-192933, filed Nov. 20, 2020, which is hereby incorporated by reference herein in its entirety.

What is claimed is:

1. An apparatus comprising:
an acquisition unit configured to acquire a first image and a second image obtained by detecting radiation of different energies, and a subtraction image generated using the first image and the second image; and
a control unit configured to control output of the first image, the second image, and the subtraction image based on examination information.

2. The apparatus according to claim 1, wherein the examination information includes information regarding use of diagnosis of the subtraction image.

3. The apparatus according to claim 2, wherein the examination information includes at least one of order information including at least one of a capturing region and a capturing direction of radiography, capturing technique information for determining whether to perform subtraction processing for generating the subtraction image, and detector information indicating a method for acquiring the first image and the second image.

4. The apparatus according to claim 1, wherein the control unit acquires output information in which the examination information is associated with information indicating which image of the first image, the second image, and the subtraction image is to be output, and controls output of the first image, the second image, and the subtraction image according to the output information.

5. An apparatus comprising:
an acquisition unit configured to acquire a first image obtained by detecting radiation of a first energy, a second image obtained by detecting radiation of a lower energy than the first energy, and a subtraction image generated using the first image and the second image; and
a control unit configured to output at least the first image and the subtraction image in a case where the subtraction image is an image in which soft tissue is emphasized, and output at least the second image and the subtraction image in a case where the subtraction image is an image in which bone is emphasized.

6. The apparatus according to claim 5, further comprising a determination unit configured to determine whether the subtraction image is an image in which soft tissue is emphasized or an image in which bone is emphasized,
wherein the control unit outputs, as a result of the determination, at least the first image and the subtraction image in a case where the subtraction image is an image in which soft tissue is emphasized, and outputs at least the second image and the subtraction image in a case where the subtraction image is an image in which bone is emphasized.

7. The apparatus according to claim 1, further comprising a providing unit configured to provide dosage information to the subtraction image.

8. The apparatus according to claim 7,
wherein the providing unit provides dosage information of an image that is not output out of the first image and the second image to the subtraction image, and
wherein the control unit outputs the subtraction image to which the dosage information of the image that is not output out of the first image and the second image is provided.

9. The apparatus according to claim 8, wherein, in a case where the first image and the second image are images acquired by one time of irradiation, the information providing unit provides dosage information in the one time of irradiation to the subtraction image.

10. A system comprising:
an apparatus including:
an acquisition unit configured to acquire a first image and a second image obtained by detecting radiation of different energies, and a subtraction image generated using the first image and the second image; and
a control unit configured to control output of the first image, the second image, and the subtraction image based on examination information and
a management system configured to store an image output from the apparatus.

11. A method comprising:
generating a subtraction image using a first image and a second image obtained by detecting radiation of different energies; and
controlling output of the first image, the second image, and the subtraction image based on examination information.

12. A method comprising:
acquiring a first image obtained by detecting radiation of a first energy, a second image obtained by detecting radiation of a lower energy than the first energy, and a subtraction image generated using the first image and the second image; and
outputting at least the first image and the subtraction image in a case where the subtraction image is an image in which soft tissue is emphasized, and outputting at least the second image and the subtraction image in a case where the subtraction image is an image in which bone is emphasized.

13. The method according to claim 12, wherein the examination information includes information regarding use of diagnosis of the subtraction image.

14. The method according to claim 12, further comprising:
acquiring output information in which the examination information is associated with information indicating which image of the first image, the second image, and the subtraction image is to be output; and
controlling output of the first image, the second image, and the subtraction image according to the output information.

15. The method according to claim 12, further comprising providing dosage information to the subtraction image.

16. A non-transitory computer-readable storage medium storing a program for causing a computer to execute a method, the method comprising:
acquiring a first image obtained by detecting radiation of a first energy, a second image obtained by detecting radiation of a lower energy than the first energy, and a subtraction image generated using the first image and the second image; and
outputting at least the first image and the subtraction image in a case where the subtraction image is an image in which soft tissue is emphasized, and outputting at least the second image and the subtraction image in a case where the subtraction image is an image in which bone is emphasized.

17. The non-transitory computer-readable storage medium according to claim 16, wherein the examination information includes information regarding use of diagnosis of the subtraction image.

18. The non-transitory computer-readable storage medium according to claim 16, further comprising:
acquiring output information in which the examination information is associated with information indicating which image of the first image, the second image, and the subtraction image is to be output; and
controlling output of the first image, the second image, and the subtraction image according to the output information.

19. The non-transitory computer-readable storage medium according to claim 16, further comprising providing dosage information to the subtraction image.
